(19) United States
(12) Patent Application Publication
(10) Pub. No.: US 2019/0298851 A1
(43) Pub. Date: Oct. 3, 2019

(54) SEQUENTIAL CLICK REACTIONS FOR THE SYNTHESIS AND FUNCTIONALIZATION OF HYDROGEL MICROSPHERES AND SUBSTRATES

(71) Applicant: The Texas A&M University System, College Station, TX (US)

(72) Inventors: Daniel L. Alge, College Station, TX (US); Faraz Jivan, College Station, TX (US); Ramanathan Yegappan, College Station, TX (US); Colin R. Dodson, College Station, TX (US)

(73) Assignee: The Texas A&M University System, College Station, TX (US)

(21) Appl. No.: 16/306,476

(22) PCT Filed: Jun. 1, 2017
(86) PCT No.: PCT/US17/35540

§ 371 Date: Nov. 30, 2018

Related U.S. Application Data
(60) Provisional application No. 62/466,895, filed on Mar. 3, 2017.
(60) Provisional application No. 62/344,055, filed on Jun. 1, 2016.

(51) Int. Cl. A61K 47/69; A61K 47/54; A61K 47/20; A61K 9/16

(52) U.S. Cl. CPC A61K 47/6903; A61K 47/6935; A61K 9/1682; A61K 47/20; A61K 47/545

(57) ABSTRACT

The present disclosure describes two click reactions to sequentially polymerize and then functionalize hydrogel microparticles to provide therapeutic agent-functionalized crosslinked hydrogel microparticles. The two click reactions have one common reactive group, and are non-interfering with respect to one another. The therapeutic agents can be released from the hydrogel microparticles over time as the hydrogel degrades, or the therapeutic agents can be released from the hydrogel microparticles by enzymatic degradation or hydrolysis. In some embodiments, the biological function of the therapeutic agents can be preserved or enhanced when covalently bound to the hydrogel microparticles. The present disclosure also provides methods for the production of hydrogel cell culture substrates. The methods overcome the hurdle of protein functionalization through the implementation of an advanced but simple chemical strategy that involves modifying a protein of interest with chemical groups (e.g., tetrazines) that are able to react with complementary chemical groups (e.g., norbornenes) immobilized within a mechanically-tunable hydrogel.

### BACKGROUND

Hydrogel microparticles are of interest as for use as tissue fillers, drug delivery systems, cell delivery systems, wound dressings and scaffolds for tissue engineering; and in applications such as drug delivery, biosensing, and tissue engineering. They can also be used as building blocks for the assembly of tissue engineering scaffolds.

While a number of methods for fabricating hydrogel microparticles have been described in the literature, free radical chain-growth polymerization is the most commonly used chemistry. Hydrogel microparticles have previously been synthesized by liquid-liquid phase separation, which can be further subdivided based on phase separation strategies: solution, suspension, or emulsion polymerizations. Various techniques, including photolithography, micromolding, droplet generators, self-assembly, and microfluidics can be used in conjunction with these polymerization strategies. The liquid-liquid two phase polymerization methods are advantageous in that they are simple to perform, however the techniques and polymerization chemistries used in tandem have the ability to improve the high throughput and scalability of these systems. Thus, batch microparticle syntheses require the need for a rapid polymerization system, such as a photochemistry-based polymerization. While acrylate-based radical polymerizations have been previously used, they tend to suffer from oxygen sensitivity during polymerization, afford less control over polymerization conditions, and produce radical-filled environments which can affect protein bioactivity.

Hydrogel cell culture substrates are also of interest, having tunable mechanical properties for the study of cell biology. Early studies, which focused on various stem cells and cancer cells, demonstrated the importance of this approach. A small group of researchers are now applying the hydrogel substrate approach to other cell types. However, most life scientists are unable to make these hydrogel materials themselves because it requires expertise in polymer science, and there are no commercially available products.

Hydrogels have become a widely used tool in fundamental studies of cell biology because these soft materials mimic the mechanical properties of tissues in the body. Importantly, studies using hydrogel substrates for cell culture have shown that the hydrogel mechanics affect myriad cell types, which has important implications because most of what is known about cell biology has been learned by studying cells on rigid plastic dishes. Hydrogel materials are revolutionizing stem cell and cancer biology, but interest is only growing. Unfortunately, the current hydrogel platforms described in the research literature are cumbersome to work with, which is impeding broader adoption of these materials.

The biggest hurdle to the adoption of hydrogel materials is protein functionalization, since these materials must be modified with tissue-derived proteins to promote the attachment of adhesive cells. Varying the protein is also of interest to many researchers, since the biochemical signals provided by the adhesive protein can influence cells, sometimes in mechanically-dependent ways.

There is a need for hydrogel microparticles that can be synthesized in a specific, efficient, high yielding manner, using mild reaction conditions. There is also a need for hydrogel materials that can be easily functionalized with proteins and that are simple to utilize. The present disclosure seeks to fulfill this need and provides further related advantages.

### SUMMARY

In one aspect, the present disclosure features a method of making hydrogel microspheres, including:

(a) providing a reaction mixture including (i) a biocompatible polymer, wherein the biocompatible polymer includes at least two of a first reactive group on each polymer, and (ii) a crosslinker comprising at least two of a second reactive group, wherein the second reactive group is reactive with the first reactive group via a first click reaction;

(b) reacting the reaction mixture to react the first and second reactive groups to provide a crosslinked hydrogel microsphere;

(c) providing a therapeutic agent including a third reactive group, wherein the third reactive group is reactive with the first reactive group via a second click reaction; and

(d) reacting the therapeutic agent with the first reactive group in the crosslinked hydrogel microsphere to provide a therapeutic agent-conjugated hydrogel microsphere.

In another aspect, the present disclosure features a crosslinked hydrogel microparticle including poly(ethylene glycol) chains crosslinked with

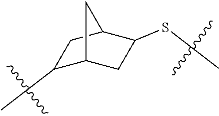 [SMILES: *SC1CC2CC1CC2*]

moieties; and

therapeutic agents-containing moieties of Formula (I)

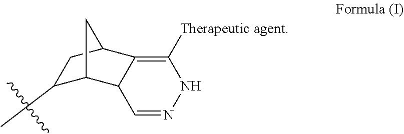

In yet one aspect, the present disclosure features a method of making a hydrogel substrate, including:

(a) providing a reaction mixture including:
(i) a biocompatible polymer, wherein the biocompatible polymer includes at least two of a first reactive group on each polymer, and
(ii) a crosslinker including at least two of a second reactive group, wherein the second reactive group is reactive with the first reactive group via a first click reaction; and

(b) reacting the reaction mixture to react the first and second reactive groups to provide a crosslinked hydrogel substrate.

In some embodiments, the method of making a hydrogel substrate further includes:

(c) providing an anchor molecule comprising a third reactive group, wherein the third reactive group is reactive with the first reactive group via a second click reaction; and

(d) reacting the anchor molecule with the first reactive group in the crosslinked hydrogel substrate to provide an anchor molecule-functionalized hydrogel substrate.

In yet another aspect, the present disclosure features a hydrogel substrate made using the methods described herein.

In yet another aspect, the present disclosure features a crosslinked hydrogel substrate including poly(ethylene glycol) chains crosslinked with

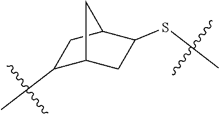 [SMILES: *SC1CC2CC1CC2*]

moieties; and anchor molecule-containing moieties of Formula (I)

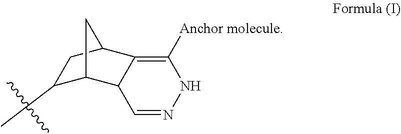

In yet another aspect, the present disclosure features a kit, including:

(a) a hydrogel substrate made using the methods described herein; and

(b) optionally an anchor molecule (e.g., a cell-adhesion molecule) including a reactive group selected from a tetrazine moiety, a hydrazine moiety, and a thiol moiety.

In yet another aspect, the present disclosure features a kit, including:

(a) a hydrogel substrate made using the methods described herein;

(b) an activated ester of 5-(4-(1,2,4,5-tetrazin-3-yl)benzylamino)-5-oxopentanoic acid; and

(c) optionally an amino (NH₂)-group-containing protein, peptide, or small molecule (e.g., optionally an amino (NH₂)-group-containing protein or peptide.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A is an illustration of the chemical structures of reactants used for the synthesis of an embodiment of polyethylene glycol (PEG) microparticles using thiol-ene click chemistry.

FIG. 1B is a schematic representation of the synthesis of an embodiment of PEG microparticles using thiol-ene click chemistry, where R₁ is a biocompatible polymer such as tetra(poly ethylene glycol), and R₂ is a thiol crosslinker (e.g., dithiothreitol).

FIG. 2A is an illustration of the chemical structures of reactants used for protein functionalization of an embodiment of microparticles using tetrazine-norbornene click chemistry.

FIG. 2B is a schematic representation of protein functionalization of an embodiment of microparticles using tetrazine-norbornene click chemistry, where R₁ is a biocompatible polymer such as tetra(poly ethylene glycol), and R₂ is a protein conjugated to tetrazine, see FIG. 2A, rightmost structure.

FIG. 3 shows a histogram of particle size distribution, a table of particle size distribution, and micrographs of an embodiment of microparticles.

FIG. 4A is a fluorescent micrograph of an embodiment of protein-functionalized microparticles, where the microparticles are functionalized with fluorescein-labeled ovalbumin conjugated to tetrazine.

FIG. 4B is a fluorescent micrograph of an embodiment of microparticles, treated with Texas-Red-labeled ovalbumin that is not conjugated to tetrazine.

FIG. 5A is a chart showing dose dependent enzyme activity of an embodiment of alkaline phosphatase (ALP)-conjugated microparticles and the bioactivity of the microparticles.

FIG. 5B is a graph showing ALP activity versus ALP concentration of an embodiment of ALP-conjugated microparticles and the bioactivity of the microparticles.

FIG. 6A is a micrograph showing ALP mineralization of an embodiment of ALP-functionalized microparticles.

FIG. 6B is a micrograph showing ALP mineralization of a control microparticle, not functionalized with ALP.

FIG. 6C is a chart showing absorbance at 405 nm of an embodiment of ALP-functionalized microparticles.

### DETAILED DESCRIPTION

Click chemistry is a versatile tool for the synthesis and functionalization of polymeric biomaterials. Click chemistry encompasses a chemical toolkit of highly efficient, specific, and high yielding reactions that can be performed under mild conditions and produce inoffensive byproducts. Click reactions can be modular; wide in scope; have simple reaction conditions; have readily available starting materials and reagents; use no solvent or one that is benign, such as water, or easily removable solvents; and/or have simple product isolation procedures. Click chemistry reactions, with their accelerated kinetics, mild reaction conditions, oxygen insensitivity, and high specificity/efficiency, can be used to provide a superior polymerization mechanism that is rapid, highly controllable, and protein-friendly. Examples of click-type reactions include copper-catalyzed azide-alkyne cycloadditions, strain-promoted azide-alkyne cycloadditions, thiol-ene additions, thiol-yne additions, thiol Michael additions, Diels-Alder reactions, and inverse electron demand Diels-Alder reactions.

Two notable click reactions that are gaining importance for biological applications are the thiol-ene and bioorthogonal tetrazine reactions. The thiol-ene click reaction refers to the radical-mediated addition of a thiol to a non-sterically hindered alkene. Key advantages of thiol-ene click reactions include their insensitivity to oxygen and the potential for rapid yet stoichiometrically controlled polymerization, which can be further kinetically controlled via photoinitiation.

Bio-orthogonal tetrazine click reactions proceed via an inverse electron-demand Diels-Alder cycloaddition between tetrazines and electron rich alkenes, such as norbornene and trans-cyclooctene. Importantly, tetrazine click reactions can proceed with fast reaction rates, do not require radical initiation, can be performed in aqueous conditions at physiologic temperature and pH, and are generally compatible with cells and biologics. Thus, tetrazine click chemistry is considered to be bioorthogonal, because these reactions can occur inside of living systems without interfering with native biochemical processes.

The present disclosure applies two click reactions to sequentially polymerize and then functionalize hydrogel microparticles (e.g., poly(ethylene glycol) (PEG)-based hydrogel microparticles). The present disclosure also applies two click reactions to sequentially polymerize and then functionalize hydrogel substrates (e.g., poly(ethylene glycol) (PEG)-based hydrogel substrates). Specifically, hydrogel microparticles and/or substrates can be fabricated using thiol-ene click chemistry and then decorated with a bioactive protein using tetrazine click chemistry.

### Definitions

At various places in the present specification, substituents of compounds of the disclosure are disclosed in groups or in ranges. It is specifically intended that the disclosure include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

It is further intended that the compounds of the disclosure are stable. As used herein “stable” refers to a compound that is sufficiently robust to survive isolation to a useful degree of purity from a reaction mixture.

It is further appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

“Optionally substituted” groups can refer to, for example, functional groups that may be substituted or unsubstituted by additional functional groups. For example, when a group is unsubstituted, it can be referred to as the group name, for example alkyl or aryl. When a group is substituted with additional functional groups, it may more generically be referred to as substituted alkyl or substituted aryl.

As used herein, the term “substituted” or “substitution” refers to the replacing of a hydrogen atom with a substituent other than H. For example, an “N-substituted piperidin-4-yl” refers to replacement of the H atom from the NH of the piperidinyl with a non-hydrogen substituent such as, for example, alkyl.

As used herein, the term “alkyl” refers to a straight or branched hydrocarbon groups. In some embodiments, alkyl has 1 to 10 carbon atoms (e.g., 1 to 8 carbon atoms, 1 to 6 carbon atoms, 1 to 3 carbon atoms, 1 or 2 carbon atoms, or 1 carbon atom). Representative alkyl groups include methyl, ethyl, propyl (e.g., n-propyl, isopropyl), butyl (e.g., n-butyl, sec-butyl, and tert-butyl), pentyl (e.g., n-pentyl, tert-pentyl, neopentyl, isopentyl, pentan-2-yl, pentan-3-yl), and hexyl (e.g., n-pentyl and isomers) groups.

As used herein, the term “alkylene” refers to a linking alkyl group.

As used herein, “alkenyl” refers to an alkyl group having one or more double carbon-carbon bonds. The alkenyl group can be linear or branched. Example alkenyl groups include ethenyl, propenyl, and the like. An alkenyl group can contain from 2 to about 50, from 2 to about 24, from 2 to about 20, from 2 to about 10, from 2 to about 8, from 2 to about 6, or from 2 to about 4 carbon atoms.

As used herein, “alkenylene” refers to a linking alkenyl group.

As used herein, “alkynyl” refers to an alkyl group having one or more triple carbon-carbon bonds. The alkynyl group can be linear or branched. Example alkynyl groups include ethynyl, propynyl, and the like. An alkynyl group can contain from 2 to about 50, from 2 to about 24, from 2 to about 20, from 2 to about 10, from 2 to about 8, from 2 to about 6, or from 2 to about 4 carbon atoms.

As used herein, “alkynylene” refers to a linking alkynyl group.

As used herein, the term “cycloalkyl” refers to non-aromatic carbocycles including cyclized alkyl, alkenyl, and alkynyl groups. Cycloalkyl groups can include mono- or polycyclic (e.g., having 2, 3 or 4 fused rings) ring systems, including spirocycles. In some embodiments, cycloalkyl groups can have from 3 to about 20 carbon atoms, 3 to about 14 carbon atoms, 3 to about 10 carbon atoms, or 3 to 7 carbon atoms. Cycloalkyl groups can further have 0, 1, 2, or 3 double bonds and/or 0, 1, or 2 triple bonds. Also included in the definition of cycloalkyl are moieties that have one or more aromatic rings fused (i.e., having a bond in common with) to the cycloalkyl ring, for example, benzo derivatives of pentane, pentene, hexane, and the like. A cycloalkyl group having one or more fused aromatic rings can be attached though either the aromatic or non-aromatic portion. One or more ring-forming carbon atoms of a cycloalkyl group can be oxidized, for example, having an oxo or sulfido substituent. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbomyl, norpinyl, norcamyl, adamantyl, and the like.

As used herein, the term “cycloalkylene” refers to a linking cycloalkyl group. As used herein, the term “perfluoroalkyl” refers to straight or branched fluorocarbon chains. In some embodiments, perfluoroalkyl has 1 to 10 carbon atoms (e.g., 1 to 8 carbon atoms, 1 to 6 carbon atoms, 1 to 3 carbon atoms, 1 or 2 carbon atoms, or 1 carbon atom). Representative alkyl groups include trifluoromethyl, pentafluoroethyl, etc.

As used herein, the term “perfluoroalkylene” refers to a linking perfluoroalkyl group.

As used herein, the term “heteroalkyl” refers to a straight or branched chain alkyl groups and where one or more of the carbon atoms is replaced with a heteroatom selected from O, N, or S. In some embodiments, heteroalkyl alkyl has 1 to 10 carbon atoms (e.g., 1 to 8 carbon atoms, 1 to 6 carbon atoms, 1 to 3 carbon atoms, 1 or 2 carbon atoms, or 1 carbon atom).

As used herein, the term “heteroalkylene” refers to a linking heteroalkyl group.

As used herein, the term “alkoxy” refers to an alkyl or cycloalkyl group as described herein bonded to an oxygen atom. In some embodiments, alkoxy has 1 to 10 carbon atoms (e.g., 1 to 8 carbon atoms, 1 to 6 carbon atoms, 1 to 3 carbon atoms, 1 or 2 carbon atoms, or 1 carbon atom). Representative alkoxy groups include methoxy, ethoxy, propoxy, and isopropoxy groups.

As used herein, the term “perfluoroalkoxy” refers to a perfluoroalkyl or cyclic perfluoroalkyl group as described herein bonded to an oxygen atom. In some embodiments, perfluoroalkoxy has 1 to 10 carbon atoms (e.g., 1 to 8 carbon atoms, 1 to 6 carbon atoms, 1 to 3 carbon atoms, 1 or 2 carbon atoms, or 1 carbon atom). Representative perfluoroalkoxy groups include trifluoromethoxy, pentafluoroethoxy, etc.

As used herein, the term “aryl” refers to an aromatic hydrocarbon group having 6 to 10 carbon atoms. Representative aryl groups include phenyl groups. In some embodiments, the term “aryl” includes monocyclic or polycyclic (e.g., having 2, 3, or 4 fused rings) aromatic hydrocarbons such as, for example, phenyl, naphthyl, anthracenyl, phenanthrenyl, indanyl, and indenyl.

As used herein, the term “arylene” refers to a linking aryl group.

As used herein, the term “aralkyl” refers to an alkyl or cycloalkyl group as defined herein with an aryl group as defined herein substituted for one of the alkyl hydrogen atoms. A representative aralkyl group is a benzyl group.

As used herein, the term “aralkylene” refers to a linking aralkyl group.

As used herein, the term “heteroaryl” refers to a 5- to 10-membered aromatic monocyclic or bicyclic ring containing 1-4 heteroatoms selected from O, S, and N. Representative 5- or 6-membered aromatic monocyclic ring groups include pyridine, pyrimidine, pyridazine, furan, thiophene, thiazole, oxazole, and isooxazole. Representative 9- or 10-membered aromatic bicyclic ring groups include benzofuran, benzothiophene, indole, pyranopyrrole, benzopyran, quionoline, benzocyclohexyl, and naphthyridine.

As used herein, the term “heteroarylene” refers to a linking heteroaryl group.

As used herein, the term “halogen” or “halo” refers to fluoro, chloro, bromo, and iodo groups.

As used herein, “alkoxy” refers to an —O-alkyl group. Example alkoxy groups include methoxy, ethoxy, propoxy (e.g., n-propoxy and isopropoxy), t-butoxy, and the like.

As used herein, “haloalkoxy” refers to an —O-(haloalkyl) group.

As used herein, “amino” refers to —NH₂.

As used herein, “thiol” refers to —SH.

As used herein, “aminooxy” refers to —ONH₂.

As used herein, the term “copolymer” refers to a polymer that is the result of polymerization of two or more different monomers. The number and the nature of each constitutional unit can be separately controlled in a copolymer. The constitutional units can be disposed in a purely random, an alternating random, a regular alternating, a regular block, or a random block configuration unless expressly stated to be otherwise. A purely random configuration can, for example, be: x-x-y-z-x-y-y-z-y-z-z-z . . . or y-z-x-y-z-y-z-x-x . . . . An alternating random configuration can be: x-y-x-z-y-x-y-z-y-x-z . . . , and a regular alternating configuration can be: x-y-z-x-y-z-x-y-z . . . . A regular block configuration (i.e., a block copolymer) has the following general configuration: . . . x-x-x-y-y-y-z-z-z-x-x-x . . . , while a random block configuration has the general configuration: . . . x-x-x-z-z-x-x-y-y-y-y-z-z-z-x-x-z-z-z- . . . .

As used herein, the term “random copolymer” is a copolymer having an uncontrolled mixture of two or more constitutional units. The distribution of the constitutional units throughout a polymer backbone (or main chain) can be a statistical distribution, or approach a statistical distribution, of the constitutional units. In some embodiments, the distribution of one or more of the constitutional units is favored.

As used herein, the term “constitutional unit” of a polymer refers to an atom or group of atoms in a polymer, comprising a part of the chain together with its pendant atoms or groups of atoms, if any. The constitutional unit can refer to a repeat unit. The constitutional unit can also refer to an end group on a polymer chain. For example, the constitutional unit of polyethylene glycol can be —CH₂CH₂O— corresponding to a repeat unit, or —CH₂CH₂OH corresponding to an end group.

As used herein, the term “repeating unit” corresponds to the smallest constitutional unit, the repetition of which constitutes a regular macromolecule (or oligomer molecule or block).

As used herein, the term “end group” refers to a constitutional unit with only one attachment to a polymer chain, located at the end of a polymer. For example, the end group can be derived from a monomer unit at the end of the polymer, once the monomer unit has been polymerized. As another example, the end group can be a part of a chain transfer agent or initiating agent that was used to synthesize the polymer.

As used herein, the term “terminus” of a polymer refers to a constitutional unit of the polymer that is positioned at the end of a polymer backbone.

As used herein, the term “cationic” refers to a moiety that is positively charged, or ionizable to a positively charged moiety under physiological conditions. Examples of cationic moieties include, for example, amino, ammonium, pyridinium, imino, sulfonium, quaternary phosphonium groups, etc.

As used herein, the term “anionic” refers to a functional group that is negatively charged, or ionizable to a negatively charged moiety under physiological conditions. Examples of anionic groups include carboxylate, sulfate, sulfonate, phosphate, etc.

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### Hydrogel Microparticles and Hydrogel Substrates

The present disclosure provides a method of making hydrogel microspheres including (a) providing a reaction mixture including (i) a biocompatible polymer, where the biocompatible polymer includes at least two of a first reactive group on each polymer, and (ii) a crosslinker including at least two of a second reactive group, where the second reactive group is reactive with the first reactive group via a first click reaction; (b) reacting the reaction mixture to react the first and second reactive groups to provide a crosslinked hydrogel microsphere; (c) providing a therapeutic agent including a third reactive group, wherein the third reactive group is reactive with the first reactive group via a second click reaction; and (d) reacting the therapeutic agent with the first reactive group in the crosslinked hydrogel microsphere to provide a therapeutic agent-conjugated hydrogel microsphere.

In some embodiments, the reaction mixture is an aqueous two-phase emulsion. In some embodiments, the aqueous two-phase emulsion includes a dextran bulk phase and a poly(ethylene glycol) dispersed phase. The volume ratio of bulk to dispersed phase can range from 15:1 to 100:1 (e.g., from 20:1 to 100:1, from 40:1 to 100:1, from 60:1 to 100:1, from 80:1 to 100:1, from 20:1 to 80:1, from 40:1 to 80:1, from 60:1 to 80:1, from 20:1 to 60:1, or from 40:1 to 60:1).

In some embodiments, in step (a), the reaction mixture further includes a photoinitiator (e.g., lithium acylphosphinate, Irgacure 2959, and/or eosin Y). In some embodiments, step (b) further includes electrospraying the reaction mixture while reacting the reaction mixture. In some embodiments, in step (b), reacting the reaction mixture includes irradiating the reaction mixture. The irradiation can have a wavelength range of 320-600 nm, have an intensity of 5-100 mW/cm² (e.g., 20-100 mW/cm², 40-100 mW/cm², 60-100 mW/cm², 80-100 mW/cm², 20-80 mW/cm², 40-80 mW/cm², or 60-80 mW/cm²), can have a duration of 1-10 min, at ambient temperature, and/or ambient pressure.

In some embodiments, the first click reaction is a photoinitiated click reaction. In some embodiments, the second click reaction is a non-photoinitiated click reaction.

In some embodiments, the first reactive group is selected from a norbornene moiety, a substituted norbornene moiety, an aromatic or aliphatic aldehyde moiety, and a C₂₋₆ alkyne moiety (e.g., a pentyne or propargyl moiety). In some embodiments, the first reactive group is a norbornene moiety

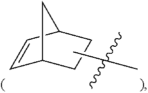

a carboxylic acid-substituted norbornene moiety

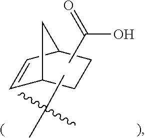

and/or a norbornene moiety substituted at a 2-

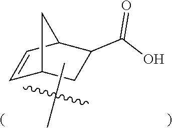

or 3-position with carboxylic acid

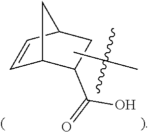

In some embodiments, the carboxylic acid-substituted norbornene moiety is reacted with the biocompatible polymer to form an ester linkage connecting the norbornene moiety with the biocompatible polymer, such that the first reactive group is an ester-substituted norbornene moiety

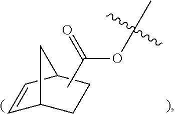

and/or a norbornene moiety substituted at a 2-

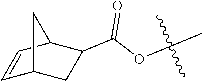 [SMILES: *OC(=O)C1CC2C=CC1C2]

or 3-position with an ester

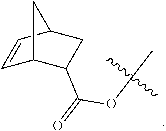 [SMILES: *OC(=O)C1CC2C=CC1C2]

In some embodiments, the second reactive group is selected from a thiol moiety (—SH), an aminooxy moiety (—O—NH₂), and an azide moiety (—N₃). In certain embodiments, the second reactive group is a thiol moiety.

In some embodiments, the crosslinker is dithiothreitol

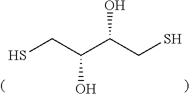

and/or PEG-di-thiol.

In any of the above-embodiments, the reaction mixture can include a first reactive group to second reactive group ratio of greater than 1 (e.g., a ratio of 1.1 or more, 1.2 or more, 1.3 or more, 1.4 or more, 1.5 or more, 1.6 or more, 1.7 or more, 1.8 or more, 1.9 or more, or 2.0 or more and/or 2.0 or less, 1.9 or less, 1.8 or less, 1.7 or less, 1.6 or less, 1.6 or less, 1.4 or less, 1.3 or less, 1.2 or less, or 1.1 or less). In some embodiments, in step (b), the second reactive groups are reacted with a portion of the first reactive groups to provide a crosslinked hydrogel microsphere including unreacted first reactive groups.

In some embodiments, the third reactive group is selected from a tetrazine moiety (6-membered aromatic ring containing 4 nitrogen atoms, such as 1,2,4,5-tetrazine

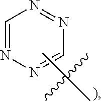

a hydrazine moiety (—NHNH₂), and a thiol moiety (—SH). In certain embodiments, the third reactive group is a tetrazine moiety. The second click reaction can occur in water, at temperatures ranging from 10° C. to 40° C. (e.g., from 10° C. to 30° C., from 20° C. to 40° C., or from 30° C. to 40° C.), have a duration of from 0.5 to 24 hours (e.g., from 1 to 24 hours, from 3 to 24 hours, from 6 to 24 hours, from 12 to 24 hours, from 1 to 12 hours, from 3 to 12 hours, or from 6 to 12 hours), and/or can occur at ambient pressure.

In some embodiments, the therapeutic agent is a protein, such as a growth factor (e.g., bone morphogenetic protein-2, bone morphogenetic protein-7, vascular endothelial growth factor, and/or nerve growth factor), an enzyme (e.g., alkaline phosphatase, and/or chondroitinase ABC), or an antibody (e.g., Trastuzumab, Bevacizumab, and/or Adalimumab); the therapeutic agent is a peptide (e.g., growth factor mimics, antineoplastic peptides, and/or immunosuppressive peptides); and/or the therapeutic agent is a small molecule therapeutic agent (e.g., doxorubicin, and/or paclitaxel). In some embodiments, the biocompatible polymer is selected from poly(ethylene glycol), hyaluronic acid, alginate, and gelatin, which can have a number average molecular weights in the range of 1,000-250,000 Da (e.g., 5 to 250 kDa, 10 to 250 kDa, 50 to 250 kDa, 100 to 250 kDa, 5 to 200 kDa, 10 to 200 kDa, 50 to 200 kDa, 100 to 200 kDa, 5 to 100 kDa, 10 to 100 kDa, or 50 to 100 kDa). In certain embodiments, the biocompatible polymer is poly(ethylene glycol), such as a branched poly(ethylene glycol). In some embodiments, the poly(ethylene glycol) is a tetra(poly(ethylene glycol)) that includes terminal norbornene moieties (e.g., norbornene moieties at one or more of the tetra(poly(ethylene glycol)) termini).

In some embodiments, the crosslinked hydrogel microparticles have an average maximum dimension of from 5 μm to 1 mm (e.g., 5 μm to 500 μm, 5 μm to 200 μm, or 5 μm to 100 μm). The average maximum dimension can be measured by analyzing bright field images of the microparticles with appropriate software such as ImageJ to determine average particle size. Fluorescence microscopy images can also be analyzed. In order to select a maximum number of particles in each image, without background interference, a lower threshold can set prior to obtaining particle diameters. At least 100 particles can be analyzed to obtain the average maximum dimension of the microparticles. Alternatively, a particle sizing device such as a Coulter Counter can be used to measure particle size.

In some embodiments, the present disclosure provides a crosslinked hydrogel microparticle, made using any one of the methods described above.

For example, the crosslinked crosslinked hydrogel microparticle can have poly(ethylene glycol) chains crosslinked with

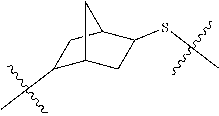 [SMILES: *SC1CC2CC1CC2*]

moieties,

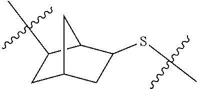 [SMILES: *SC1CC2CC(*)C1C2]

moieties,

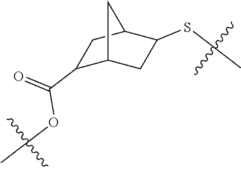 [SMILES: *OC(=O)C1CC2CC1CC2S*]

moieties, and/or

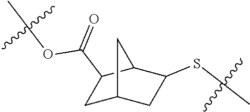 [SMILES: *OC(=O)C1CC2CC(S*)C1C2]

moieties; and

therapeutic agents-containing moieties of Formula (I)

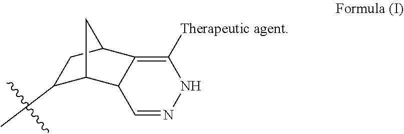

As another example, the crosslinked crosslinked hydrogel microparticle can have poly(ethylene glycol) chains crosslinked with

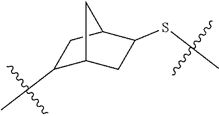 [SMILES: *SC1CC2CC1CC2*]

moieties, and/or

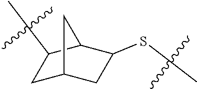 [SMILES: *SC1CC2CC(*)C1C2]

moieties; and

therapeutic agents-containing moieties of Formula (I)

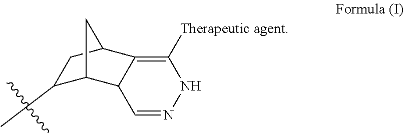

As another example, the crosslinked crosslinked hydrogel microparticle can have poly(ethylene glycol) chains crosslinked with

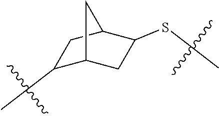 [SMILES: *SC1CC2CC1CC2*]

moieties; and

therapeutic agents-containing moieties of Formula (I)

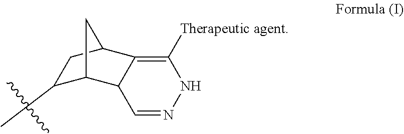

### Hydrogel Substrates

As discussed above, thiol-ene click chemistry is advantageous because of its fast kinetics and amenability to photopolymerization, whereas tetrazine click chemistry can exploit unreacted alkenes in the network for bioorthogonal protein conjugation. Thiol-ene click chemistry can provide rapid hydrogel substrate polymerization. Furthermore, by using a [thiol]:[norbornene] ratio of less than 1, unreacted norbornene functional groups in the hydrogel can be available for protein conjugation via tetrazine click chemistry, which proceeds via an inverse electron demand Diels-Alder reaction between 1,2,4,5-tetrazines with electron rich dienophiles like norbornene.

The present disclosure provides hydrogel cell culture substrates and methods for the production of hydrogel cell culture substrates. The methods overcome the hurdle of protein functionalization through the implementation of an advanced but simple chemical strategy that involves modifying a protein of interest with chemical groups (e.g., tetrazines) that are able to react with complementary chemical groups (e.g., norbornenes) immobilized within a mechanically-tunable hydrogel having an elastic moduli, E, of 100 Pa to 500 kPa. In some embodiments, the elastic modulus is 100 Pa or more (e.g., 500 Pa or more, 1 kPa or more, 10 kPa or more, 100 kPa or more, 250 kPa or more, or 400 kPa or more) and/or 500 kPa or less (e.g., 400 kPa or less, 250 kPa or less, 100 kPa or less, 10 kPa or less, 1 kPa or less, or 400 Pa or less). Tuning of mechanical properties can be performed by, for example, varying the polymer concentration (e.g., 4 wt % to 20 wt %, 5 wt % to 20 wt %, 5 wt % to 15 wt %, or 5 wt % to 10 wt % of the total reaction mixture) and crosslinker stoichiometry (e.g., r=0.5-1.0, where “r” refers to a stoichiometric ratio of reactive group in a step growth polymerization reaction. By definition, r is 1.0 or less. For example, if 1 mol of tetrafunctional PEG-norbornene is reacted with 1 mol of dithiothreitol, then r=0.5). The hydrogel can be produced using a highly efficient photopolymerization strategy that is amenable to scale up and mass production. Protein modification can be carried out using commercially available reagents such as succinimidyl esters that react with amine groups on proteins. For example, a solution of modified protein can be added to the surface of a hydrogel to functionalize the hydrogel with the protein. The entire process from hydrogel polymerization to protein functionalization is quick and easy to perform.

In some embodiments, the methods can be used to mass produce easy-to-use kits that can be used, for example, for studying mechanical signals in cell biology, or for studying the interplay between biochemical and mechanical cues in cell biology.

The hydrogels of the present disclosure can be part of kits of pre-packaged hydrogel substrates and protein conjugation reagent. In some embodiments, the proteins are provided with the kit. In some embodiments, the proteins are provided separately from the kit.

### Hydrogel Synthesis

The present disclosure features a method of making a hydrogel substrate, including (a) providing a reaction mixture including (i) a biocompatible polymer, wherein the biocompatible polymer includes at least two of a first reactive group on each polymer, and (ii) a crosslinker comprising at least two of a second reactive group, wherein the second reactive group is reactive with the first reactive group via a first click reaction; and (b) reacting the reaction mixture to react the first and second reactive groups to provide a crosslinked hydrogel substrate. In some embodiments, the method further includes (c) providing a cell-adhesive anchor molecule including a third reactive group, wherein the third reactive group is reactive with the first reactive group via a second click reaction; and (d) reacting the anchor molecule with the first reactive group in the crosslinked hydrogel substrate to provide an anchor molecule-functionalized hydrogel substrate. As used herein, an anchor molecule is a molecule that promotes cell adhesion (e.g., a cell-adhesion molecule) to the hydrogel substrate.

In some embodiments, the reaction mixture is provided between an adhesive surface and a non-adhesive surface. An adhesive surface can be, for example, a thiolated glass coverslip. In some embodiments, the non-adhesive surface comprises polydimethylsiloxane or siliconized glass.

In some embodiments, in step (a), the reaction mixture further comprises a photoinitiator, such as lithium acylphosphinate, Irgacure 2959, and/or eosin Y.

In some embodiments, the first click reaction is a photoinitiated click reaction. In some embodiments, the second click reaction is a non-photoinitiated click reaction.

In some embodiments, in step (b), reacting the reaction mixture includes irradiating the reaction mixture. The irradiation can have a wavelength range of 320-600 nm, have an intensity of 5-100 mW/cm² (e.g., 20-100 mW/cm², 40-100 mW/cm², 60-100 mW/cm², 80-100 mW/cm², 20-80 mW/cm², 40-80 mW/cm², or 60-80 mW/cm²), can have a duration of 1-10 min, at ambient temperature, and/or ambient pressure.

In some embodiments, the first reactive group is selected from a norbornene moiety, a substituted norbornene moiety, an aromatic or aliphatic aldehyde moiety, and a C₂₋₆ alkyne moiety (e.g., a pentyne or propargyl (—CH₂C═CH) moiety). In certain embodiments, the first reactive group is a norbornene moiety

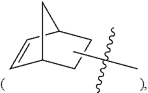

a carboxylic acid-substituted norbornene moiety

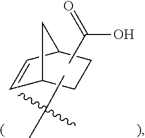

and/or a norbornene moiety substituted at a 2-

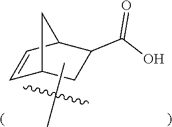

or 3-position with carboxylic acid

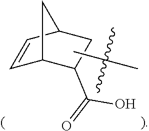

In some embodiments, the carboxylic acid-substituted norbornene moiety is reacted with the biocompatible polymer to form an ester linkage connecting the norbornene moiety with the biocompatible polymer, such that the first reactive group is an ester-substituted norbornene moiety

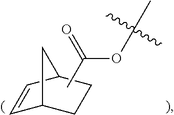

and/or a norbornene moiety substituted at a 2-

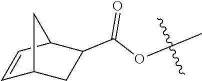 [SMILES: *OC(=O)C1CC2C=CC1C2]

or 3-position with an ester

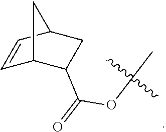 [SMILES: *OC(=O)C1CC2C=CC1C2]

In some embodiments, the second reactive group is selected from a thiol (—SH) moiety, an aminooxy (—ONH₂) moiety, and an azide (—N₃) moiety. In certain embodiments, the second reactive group is a thiol moiety.

In some embodiments, the crosslinker is dithiothreitol

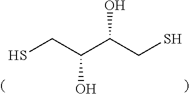

and/or PEG-di-thiol.

In some embodiments, the reaction mixture includes a first reactive group to second reactive group ratio of greater than 1 (e.g., a ratio of 1.1 or more, 1.2 or more, 1.3 or more, 1.4 or more, 1.5 or more, 1.6 or more, 1.7 or more, 1.8 or more, 1.9 or more, or 2.0 or more and/or 2.0 or less, 1.9 or less, 1.8 or less, 1.7 or less, 1.6 or less, 1.6 or less, 1.4 or less, 1.3 or less, 1.2 or less, or 1.1 or less). In some embodiments, in step (b), the second reactive groups is reacted with a portion of the first reactive groups to provide a crosslinked hydrogel substrate comprising unreacted first reactive groups.

In some embodiments, the third reactive group is selected from a tetrazine (6-membered aromatic ring containing 4 nitrogen atoms, such as 1,2,4,5-tetrazine) moiety, a hydrazine (—NHNH₂) moiety, and a thiol moiety. In some embodiments, the third reactive group is a tetrazine moiety. The second click reaction can occur in water, at temperatures ranging from 10° C. to 40° C. (e.g., from 10° C. to 30° C., from 20° C. to 40° C., or from 30° C. to 40° C.), have a duration of from 0.5 to 24 hours (e.g., from 1 to 24 hours, from 3 to 24 hours, from 6 to 24 hours, from 12 to 24 hours, from 1 to 12 hours, from 3 to 12 hours, or from 6 to 12 hours), and/or can occur at ambient pressure.

In some embodiments, the anchor molecule (e.g., a cell-adhesion molecule) is a protein (e.g., fibronectin, type I collagen, and/or laminin) and/or a peptide (e.g., having an amino acid sequence such as RGDS, IKVAV, YIGSR, GFOGER, and/or RRETAWA). In some embodiments, the biocompatible polymer is selected from poly(ethylene glycol), hyaluronic acid, alginate, and gelatin, which can have a number average molecular weights in the range of 1,000-250,000 Da (e.g., 5 to 250 kDa, 10 to 250 kDa, 50 to 250 kDa, 100 to 250 kDa, 5 to 200 kDa, 10 to 200 kDa, 50 to 200 kDa, 100 to 200 kDa, 5 to 100 kDa, 10 to 100 kDa, or 50 to 100 kDa). In certain embodiments, the biocompatible polymer is poly(ethylene glycol), for example, a branched poly(ethylene glycol). The poly(ethylene glycol) can be a tetra(poly(ethylene glycol)) including one or more (e.g., up to 4) terminal norbornene moieties (e.g., norbornene moieties at one or more of the tetra(poly(ethylene glycol)) termini).

The present disclosure also features a crosslinked hydrogel substrate made using the any one of the methods above. For example, the crosslinked hydrogel substrate can include poly(ethylene glycol) chains crosslinked with

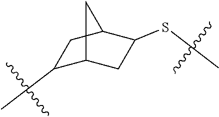 [SMILES: *SC1CC2CC1CC2*]

moieties,

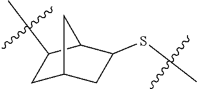 [SMILES: *SC1CC2CC(*)C1C2]

moieties,

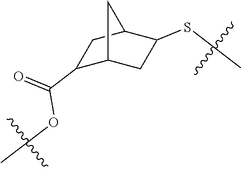 [SMILES: *OC(=O)C1CC2CC1CC2S*]

moieties, and/or

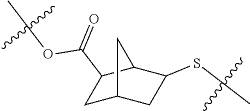 [SMILES: *OC(=O)C1CC2CC(S*)C1C2]

moieties; and anchor molecule-containing moieties of Formula (II)

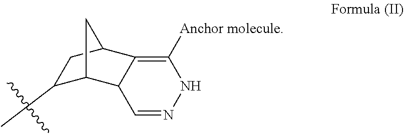

As another example, the crosslinked hydrogel substrate can have poly(ethylene glycol) chains crosslinked with

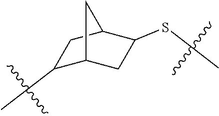 [SMILES: *SC1CC2CC1CC2*]

moieties, and/or

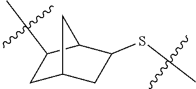 [SMILES: *SC1CC2CC(*)C1C2]

moieties; and anchor molecule-containing moieties of Formula (II)

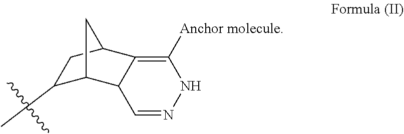

As another example, the crosslinked hydrogel substrate can have poly(ethylene glycol) chains crosslinked with

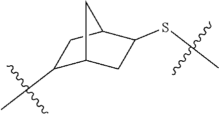 [SMILES: *SC1CC2CC1CC2*]

moieties; and anchor molecule-containing moieties of Formula (II)

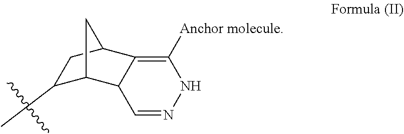

In some embodiments, poly(ethylene glycol) (PEG) hydrogels, which are widely regarded as a “blank slate” material for cell culture, are photopolymerized using norbornene-functionalized PEG star polymers and a di-thiol crosslinker (e.g., dithiothreitol, PEG-di-thiol), as described, for example, in Fairbanks, B. D. et al., Adv. Mater. 2009, 21 (48), 5005-10; Lin, C. C. et al., Biomaterials 2011, 32 (36), 9685-9695; Lin, C. C. et al., J. Appl. Polym. Sci. 2015, 132(8), 41563, herein incorporated by reference in their entireties. The mechanical properties of these hydrogels can be tuned over a physiologically relevant range using established techniques, as known to a person of ordinary skill in the art.

In some embodiments, rather than a branched PEG polymer (e.g., a star PEG), the PEG can be linear, or a mixture of linear and branched PEG polymer.

In some embodiments, the hydrogels can be prepared between an adhesive surface, such as a thiolated glass coverslip prepared using routine silane chemistry, and a non-adhesive material, such as a silanized or siliconized glass or polydimethylsiloxane. Siliconization can be performed using reagents such as Sigmacote®, available from Signma Aldrich Co. LLC.

In some embodiments, a stoichiometric excess of norbornene groups is used in the hydrogel formulation. This process, which can be completed in minutes and is scalable, can result in a flat hydrogel substrate that is attached to a rigid material, such as a glass coverslip or plastic dish, to facilitate handling.

In some embodiments, the hydrogels of the present disclosure are prepared under sterile conditions or sterilized after manufacture (e.g., by treating the hydrogel with isopropanol, gamma rays, etc.). The sterile hydrogels can then be placed into an optionally hydrated container and packaged for shipment. In some embodiments, antibiotics can be added as necessary to decrease the likelihood of (e.g., prevent) bacterial contamination and maximize shelf-life. In some embodiments, the sterilization and packaging can be done at a manufacturing facility.

### Kits

In some embodiment, the present disclosure features a kit, including (a) a hydrogel substrate made using any of the methods above, and (b) optionally a cell-adhesion molecule including a reactive group selected from a tetrazine moiety, a hydrazine moiety, and a thiol moiety.

In some embodiment, the present disclosure features a kit, including (a) a hydrogel substrate made using any of the methods above, and (b) a cell-adhesion molecule including a reactive group selected from a tetrazine moiety, a hydrazine moiety, and a thiol moiety.

In some embodiments, the present disclosure features a kit, including (a) a hydrogel substrate made using any of the methods above, (b) an activated ester of 5-(4-(1,2,4,5-tetrazin-3-yl)benzylamino)-5-oxopentanoic acid; and (c) optionally an amino-group-containing protein or peptide. In certain embodiments, the activated ester of 5-(4-(1,2,4,5-tetrazin-3-yl)benzylamino)-5-oxopentanoic acid is a succinimidyl ester of 5-(4-(1,2,4,5-tetrazin-3-yl)benzylamino)-5-oxopentanoic acid.

In some embodiments, the present disclosure features a kit, including (a) a hydrogel substrate made using any of the methods above, (b) an activated ester of 5-(4-(1,2,4,5-tetrazin-3-yl)benzylamino)-5-oxopentanoic acid; and (c) an amino-group-containing protein or peptide. In certain embodiments, the activated ester of 5-(4-(1,2,4,5-tetrazin-3-yl)benzylamino)-5-oxopentanoic acid is a succinimidyl ester of 5-(4-(1,2,4,5-tetrazin-3-yl)benzylamino)-5-oxopentanoic acid.

In some embodiments, the succinimidyl ester of 5-(4-(1,2,4,5-tetrazin-3-yl)benzylamino)-5-oxopentanoic acid (Tz-NHS) are synthesized, which is easy to produce in gram-scale quantities as described, for example, in Alge, D. L. et al., Tetrahedron Lett. 2013, 54 (41), 5639-5641, herein incorporated by reference in its entirety. The succinimidyl ester can be packaged as a solid in a container, such as a microtube. In some embodiments, the succinimidyl ester is stored cold and shipped on ice to ensure preservation of the Tz-NHS (i.e., decrease the likelihood of decomposition of Tz-NHS).

In some embodiments, protein functionalization of the hydrogels can be performed by a skilled practitioner after receipt of the kit. The protein can be provided along with the hydrogels and Tz-NHS as part of the kit, or can be obtained separately. In some embodiments, an aliquot of Tz-NHS can be reconstituted in sterile biotechnology grade dimethylsulfoxide (DMSO) and added to an appropriately buffered sterile aqueous solution of protein to provide a tetrazine-modified protein. The tetrazine functionalization of protein can take about an hour or less at room temperature. Instructions for protein functionalization, such as volumes, protein concentrations, and times can be provided in the kit.

In some embodiments, once the tetrazine-functionalized protein is obtained, the functionalized protein can be contacted with the hydrogel substrates in a tissue culture well plate. The hydrogel substrates can be incubated (e.g., in a cell culture incubator) at 37° C. for about one hour. After incubation, the hydrogels can be washed with a solution, such as an aqueous buffer, to remove unconjugated protein. The protein-functionalized hydrogels can be seeded a cell type of interest.

The following examples are included for the purpose of illustrating, not limiting, the described embodiments.

Example 1 describes the synthesis and characterization of examples of hydrogel microparticles of the present disclosure. Example 2 describes the synthesis and characterization of examples of hydrogel substrates of the present disclosure.

### EXAMPLES

### Example 1. Hydrogel Microparticle

A synthetic hydrogel microparticle platform that could be versatile and easily bioactively functionalized is described. Here, a sequential approach of thiol-norbornene and tetrazine-norbornene click chemistry results in rapidly-formed PEG hydrogel microparticles that are protein-functionalized. The major benefit of this sequential click reaction is that both chemistries rely on norbornene as a reactant and can be stoichiometrically controlled. The step-growth thiol-ene photopolymerization can be independently used to synthesize microparticles and the subsequent tetrazine-norbornene reaction can be used to tether the biomolecule of interest to microparticles. While simultaneous tethering and microparticle photopolymerization is possible with thiol-ene chemistry, thiolating the desired protein runs the risk of harming sensitive biomolecules through unwanted exposure to ultraviolet light and radicals during polymerization. The step-growth, thiol-norbornene reaction affords the use of rapid radical photopolymerization of microparticles, in the absence of biomolecules. Additionally, by performing the thiol-ene reaction off-stoichiometry (0.75:1.0 thiol-to-ene ratio), 25% of the norbornene functionalities are available to participate in the tetrazine-norbornene reaction. The non-radical initiated bioorthogonal tetrazine-norbornene click reaction can then be used to chemically conjugate the desired protein to the remaining norbornene groups without concern for compromised bioactivity. Here, PEG concentration was varied to demonstrate its effect on microparticle size. Efficacy of the sequential reactions and conjugation efficiency was tested using a tetrazine-conjugated, model fluorescent protein ovalbumin. After confirmation, versatility and bioactivity of the desired biomolecule was tested with both alkaline phosphatase and glucose-oxidase. Taken together, this study demonstrates the superiority, control, and versatility of a click chemistry-based approach for synthesizing a polymeric microparticle-protein conjugate system that is rapid, facile and bioorthogonal.

4-Arm PEG-Tetra Norbornene (PEG-NB) Synthesis

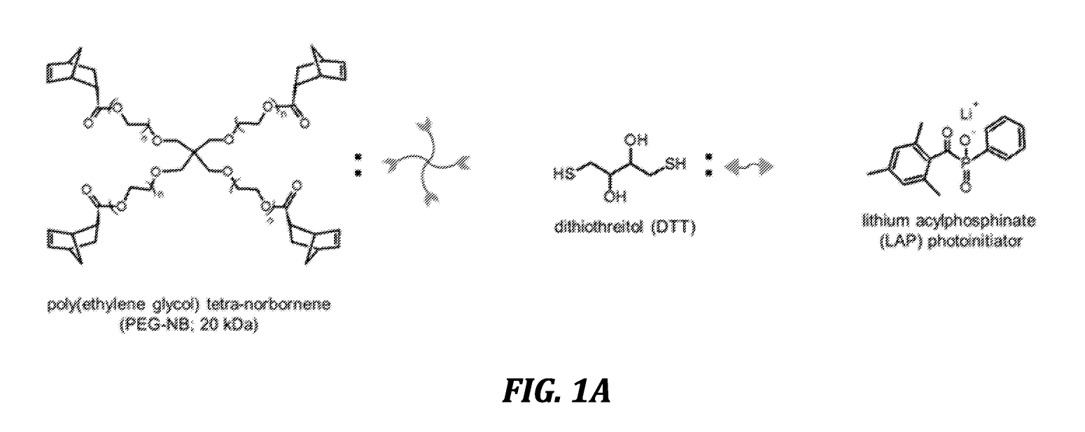

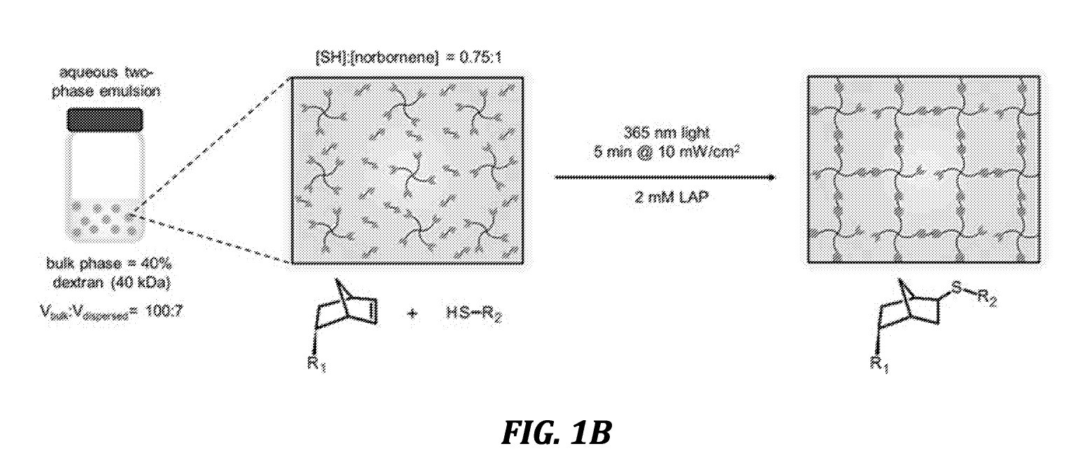

Briefly, 5-norbornene-2-carboxylic acid (10 COOH:1 PEG-OH, Alfa Aesar), diisopropylcarbodiimde (Alfa Aesar), and anhydrous dichloromethane (15 mL, Acros) were mixed for 30 min in reaction vessel under argon. Separately, 5.0 g PEG macromer (20 kDa, 4-arm PEG-hydroxyl, JenKem), 4-(dimethylamino)pyridine (0.5× to PEG-OH, Sigma Aldrich), pyridine (5× to PEG-OH, Sigma Aldrich), and anhydrous dichloromethane (20 mL) were dissolved in round bottom flask under argon. Dissolved contents of reaction vessel were filtered to remove precipitate triethylamine salts, and added to the previously-mentioned round bottom flask. Solution was mixed overnight, precipitated in 10-fold vol. excess of diethyl ether (Thermo Fisher), and vacuum filtered to yield a white precipitate (PEG-NB, see FIG. 1A). The PEG-NB was dried under vacuum for 24 hrs, dialyzed for 2 days (MWCO=10 KDa), and lyophilized to achieve quantitative yield of purified white powder, which was confirmed via NMR. ¹H NMR (300 MHz, CDCl₃) δ6.22-5.92 (1H, m), 4.30-4.16 (1H, m), 3.71-3.60 (227H, m).

PEG Microparticle Preparation and Characterization

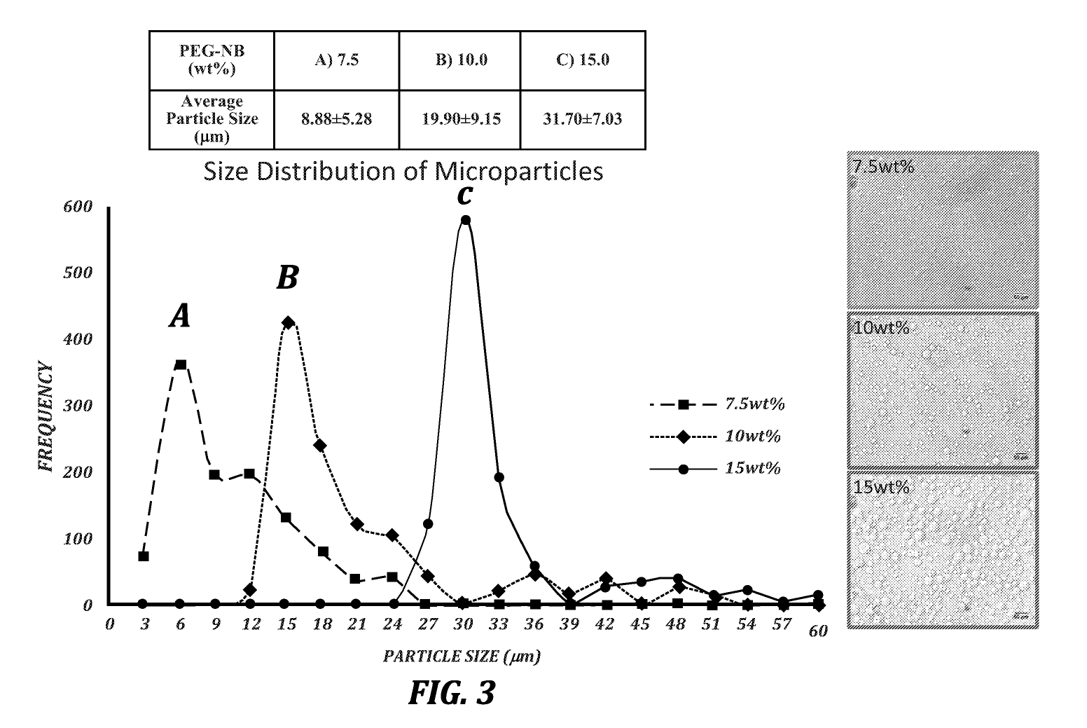

Microparticles were synthesized via an aqueous two phase emulsion system based on bulk phase dextran-disperse phase PEG polymer immiscibility (FIG. 1B). Briefly, a disperse phase consisting of varying wt % PEGNB macromer (7.5 wt %, 10 wt %, 15 wt %), dithiothreitol (0.75:1.0 thiol-ene, Alfa Aesar) and lithium phenyl-2,4,6-trimethylbenzoylphosphinate (LAP, 2 mM, see FIG. 1B), was emulsified in a bulk phase of dextran (40 wt %, 40 kDa, BioChemica) with LAP (3.125 mM). Emulsions were vortexed and allowed to sit for 10 min to allow for phase equilibria and photopolymerized with UV-365 nm light (10 mWcm², 5 min, Lumen Dynamics Omnicure S2000 Series). Microparticles were pelleted via centrifugation (4400 rpm, 10 min) in PBS (30:1 by vol.). Supernatant was decanted and microparticles were viewed on slide using bright field microscopy (10×, Nikon Eclipse TE2000-S). Microparticle diameter from bright field images were analyzed using ImageJ Particle Analyzer tool (NIH), and a size distribution histogram was plotted for varying PEG concentrations (FIG. 3). PEG-NB 10 wt % was used for subsequent experiments.

Tetrazine-Functionalized Fluorescein Ovalbumin

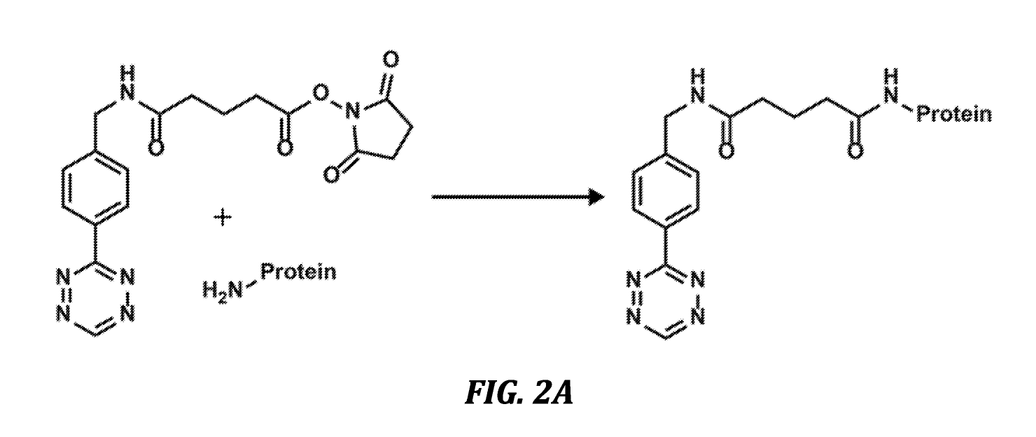

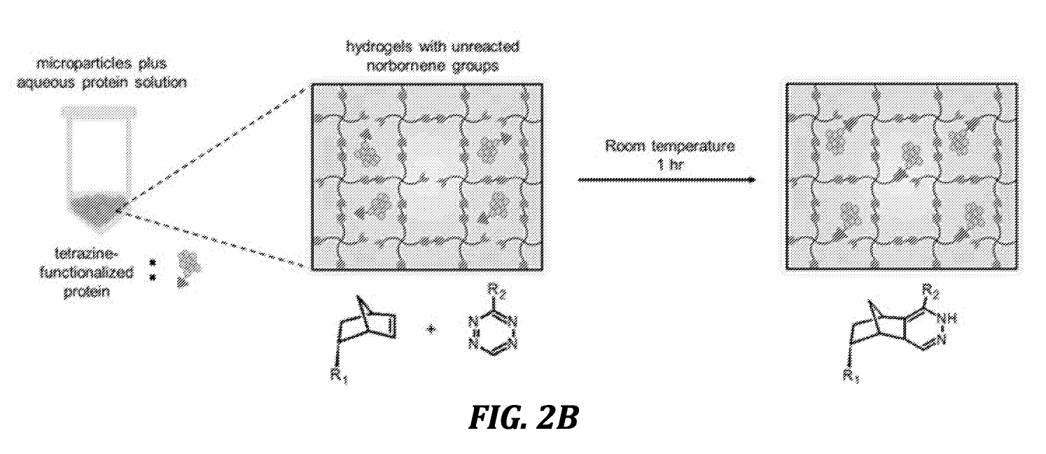

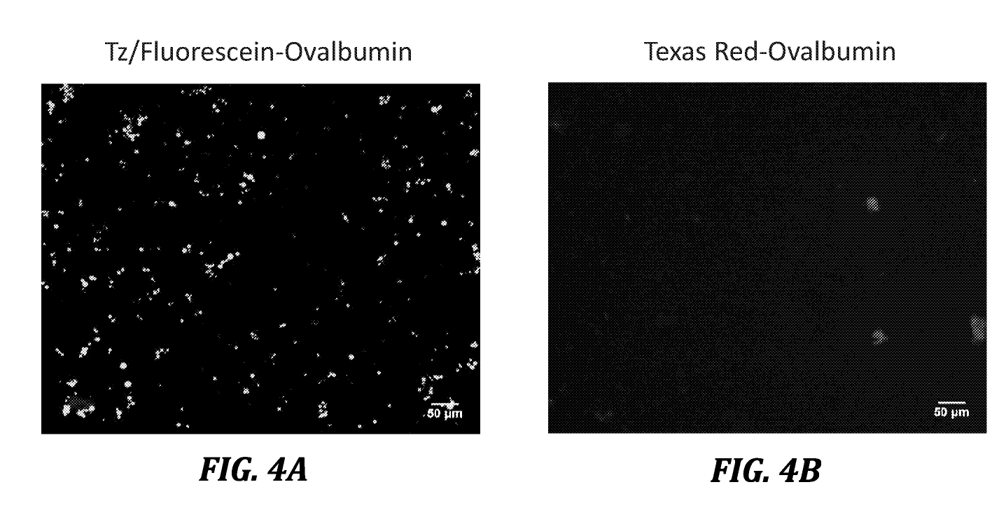

Tetrazine-NHS ester (100 mM), was added to fluorescein-ovalbumin (5 mg/mL, Life Technologies) in PBS, and allowed to incubate while covered at room temperature for 1 hour to allow for conjugation (Tz-Ovalbumin). See, FIG. 2A. Subsequent functionalization to the microparticles was achieved by incubating pelleted microparticles with Tz-ovalbumin at room temperature for 1 hour (FIG. 2B). Microparticles were washed three times with PBS (10,000 rpm, 5 min) to remove any unconjugated Tz-Ovalbumin. Referring to FIG. 4A, microparticle pellet was resuspended in PBS (600 μL) and microparticles were imaged with fluorescence microscopy (Nikon Eclipse TE2000-S). Referring to FIG. 4B, similar incubation with the microparticle pellet was performed using Texas Red-Ovalbumin (Life Technologies) without tetrazine functionalization (NF-Ovalbumin), which served as a negative control for protein tethering with tetrazine-norbornene click reaction.

Tetrazine-Functionalized Alkaline Phosphatase (Tz-ALP)

Alkaline phosphatase from bovine intestinal mucosa (Sigma Aldrich) was dissolved in PBS (50 mg/mL). Similar procedure (as above) was performed for tetrazine functionalization (Tz-ALP) and microparticle conjugation. Negative control for microparticle conjugation was also prepared with non-functionalized ALP (NF-ALP).

ALP Bioactivity

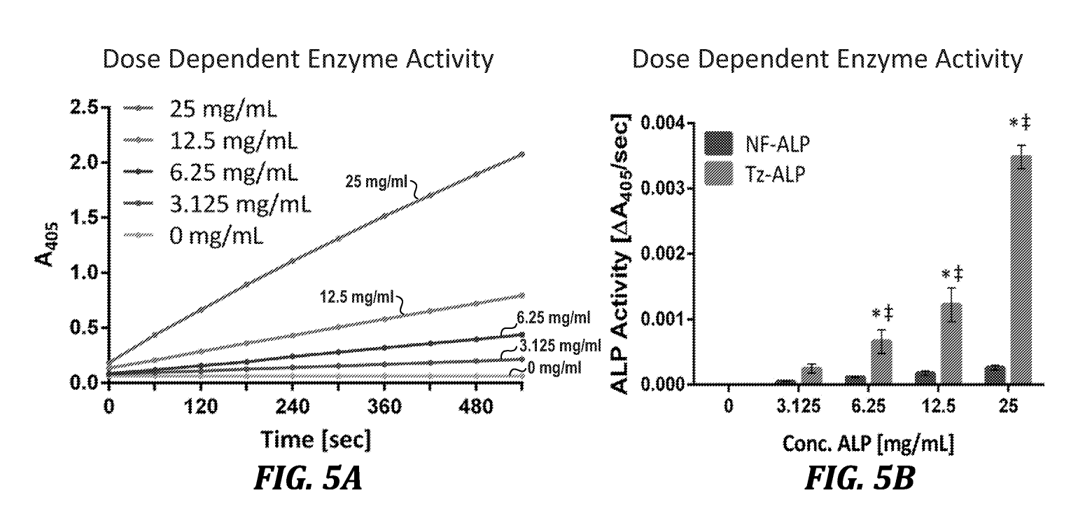

Microparticles were loaded with varying amounts of ALP to study dose-dependent bioactivity. Varying concentrations of ALP (ranging from 0 mg/mL-25 mg/mL) were conjugated to microparticles using previously mentioned protocol (see above, Tetrazine-Functionalized Alkaline Phosphatase (Tz-ALP)). Microparticles were added to para-Nitrophenylphosphate substrate (pNPP, Sigma Aldrich) in 96 well plate, as triplicates. Plate reader absorbance kinetic cycle at 405 nm was performed to detect colormetric reaction with substrate over time (FIG. 5A). Absorbance increase over time was plotted and slopes of data sets were compared to determine ALP activity (FIGS. 5A and 5B).

ALP-Induced Mineralization

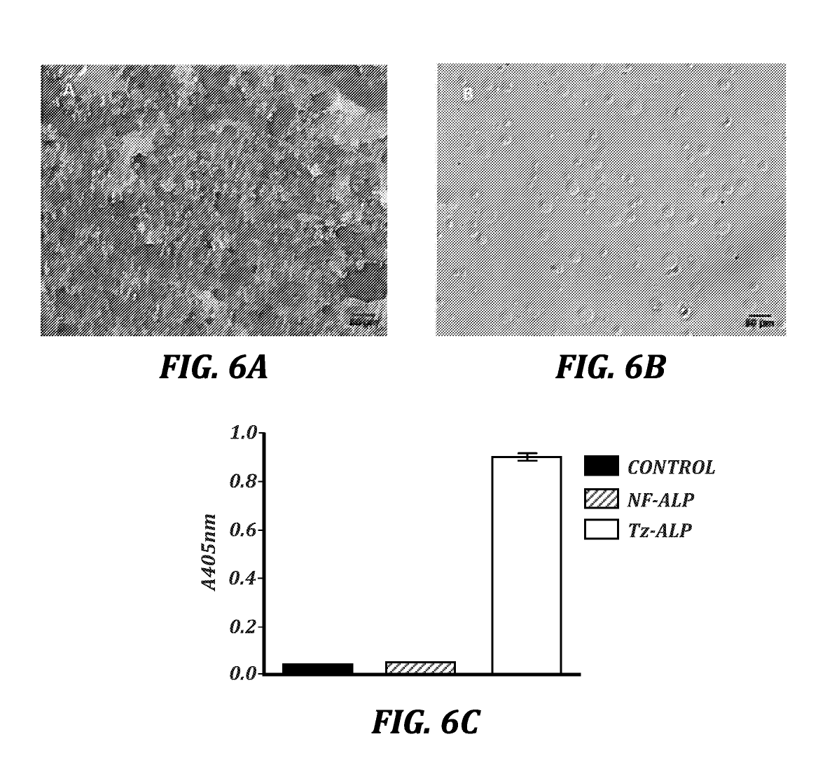

Calcium glycerophosphate (Alfa Aesar) was dissolved in milli-Q water (0.1M) and incubated with both Tz-ALP conjugated microparticles and NF-ALP microparticles (FIG. 6A), along with calcium glycerophosphate control (FIG. 6B), at 37° C. for 1 hour under 5% CO₂. Microparticles were washed three times with milli-Q water (10,000 rpm, 5 min) prior to mineralization staining. Alazarin red S (2%, pH 4.2, Electron Microscopy Sciences) was filtered through 25 mm syringe tip, nylon membrane filter (0.45 μm) and incubated with aforementioned microparticles at room temperature for 10 min. Microparticles were washed three times in milli-Q water and visualized in bright field for red-stained calcium deposits using epifluorescent microscope (Nikon Eclipse TE2000S). To quantify Alazarin red S staining for mineralization, microparticles were de-stained by incubating with 10% glacial acetic acid at room temperature for 30 min, then pelleting (10,000 rpm, 5 min) and collecting the supernatant. 10% ammonium hydroxide was added to supernatant to return the red color and acidic pH (4.1-4.5). Samples were plated as triplicates onto a 96 well plate and plate reader was used to measure absorbance at 405 nm (FIG. 6C).

Choosing Thiol-Ene Photochemistry and Emulsion-Based Microparticle Synthesis

Three of the key advantages to using the thiol-ene click chemistry photopolymerization in conjunction with the emulsion-based microparticle synthesis, are the rapid gelation times, to stoichiometric control, and high-throughput. Thiol-ene step-growth gelation kinetics and bulk storage modulus were shown to be rapid and UV-dose dependent, respectively. UV intensity and time (10 mW/cm², 5 minutes) was selected as the polymerization conditions for its mild intensity, rapid polymerization kinetics, and sufficient working time.

Showing Microparticle Size Control with PEG Concentration

User control over microparticle size using the thiol-ene polymerization and polymer immiscibility emulsion is shown. Microparticles were synthesized with different concentrations of PEG-NB macromer (7.5 wt %, 10 wt %, 15 wt %) and bright field images were analyzed in ImageJ to determine average particle size of these different batches. In order to select a maximum number of particles in each image, without background interference, a lower threshold was set prior to obtaining particle diameters. A total of 1124 particles were analyzed for each PEG-NB wt % variation. The data showed that as PEG-NB wt % increased, average particle size also increased linearly from 8.88 μm 5.28 μm to 31.7 μm±7.03 μm (FIG. 3). The increase in particle size could be attributed directly to an increase in PEG content, which increases the volume of polymer as well as the total volume of water in the microparticles at equilibrium swelling.

Polymer size is of particular importance in many aspects of tissue engineering and 3-D cell scaffolding where these particles could be effective vehicles for culturing cells within a controllable microenvironment. While successively increasing PEG concentration may not be feasible or may prove difficult to work with, further particle size control could be obtained by changing the PEG molecular weight or changing the length of the di-thiol cross linker to regulate hydrogel cross linking density and swelling. The addition of enzymatically degradable crosslinkers (i.e. matrix metalloprotease (MMP)-degradable peptides) could also enhance the functionality of these microparticles for dual therapeutic effects (one drug physically encapsulated while a second drug is tethered). Thus, future work will focus on increasing average particle size on the order of 100 μm to be more relevant in vitro and in vivo applications. Additionally, while the polydispersity of the microparticles did not affect other aspects of this project, a more monodisperse batch could be explored by filtration or drop-wise polymerization using a water-in-oil emulsion, although this may reduce high throughput and scalability.

Selective Conjugation with Tetrazine-Norbornene Click Reaction

To demonstrate the efficacy of stoichiometric control during microparticle polymerization as well as selectivity of the tetrazine-norbornene click reaction, a model tetrazine-conjugated fluorescein ovalbumin was used to visualize microparticle biotethering (FIG. 4A). Microparticles are clearly seen fluorescing green indicating fluorescein ovalbumin tethering. Alternatively, non-functionalized Texas Red ovalbumin (FIG. 4B) is only residually present, as seen by the lack of red fluorescence, as most of the unconjugated fluorophore is removed during PBS washing steps.

ALP Bioactivity

To characterize the efficiency of ALP tethering to the microparticles, supernatant from washing steps were tested for presence of Tz-ALP. As previously mentioned, one of the advantages for utilizing the bio-orthogonal tetrazine-norbornene click reaction is the lack of radical production during polymerization which can affect protein bioactivity. Thus, ALP bioactivity was confirmed. Tz-ALP micro particle bioactivity showed a concentration dependent increase from 0 mg/mL to 25 mg/mL (FIGS. 5A and 5B). Bioactivity of 6.25 mg/mL Tz-ALP and higher concentrations were statistically significant (p<0.05) when compared to their negative control NF-ALP counterparts (*), as well as between all other concentrations of Tz-ALP(∀) (FIGS. 5A and 5B).

Tz-ALP Microparticle-Induced Mineralization

Mineralization was quantified to demonstrate the biomineralization and nucleation effect of actively tethered ALP. Tz-ALP microparticles (FIG. 6A) induced mineralization in calcium glycerophosphate significantly more than NF-ALP or calcium glycerophostate alone (FIG. 6B), as seen by the drastic difference in absorbance after de-staining (FIG. 6C). From visualization of the alizarin red S staining, Tz-ALP micro particles seem to act as nucleation centers by allowing mineralization to start from the microparticle surface and bud-off into solution.

In conclusion, sequential thiol-ene and tetrazine-norbornene click reactions allowed for rapid and stoichiometrically-controlled microparticle polymerization with bio-active protein tethering is demonstrated. The results show facile control of microparticle size and maintenance of protein bioactivity/function after conjugation. The polymerization and tethering mechanisms used, address the need for versatile, user-controlled chemical tools for synthesizing bioactive polymers. Taken together, this strategy of bioactive functionalization can be employed for theoretically any protein and shows promise for tissue engineering applications in vitro and noninvasive.

### Example 2. Hydrogel Substrates

PEG hydrogels were fabricated by thiol-norbornene photopolymerization using a 4 arm, 20 kDa PEG-norbornene precursor and dithiothrietol at a thiol to norbornene ([SH]:[norbornene]) ratio of 0.75:1. The hydrogels were covalently bonded to silane functionalized circular glass coverslips. Subsequently, 10 molar equivalents of Tz-NHS were used to modify fluorescein-labelled bovine serum albumin (BSA). The tetrazine/flourescein-modified BSA was pipetted on top of the hydrogels and incubated for 1 h.

After washing off unbound protein, the hydrogels were imaged by confocal fluorescence microscopy, which revealed a uniform surface coating of fluorescein-BSA with slight diffusion of the protein into the bulk of the gel. The same procedure was then used to functionalize PEG hydrogels with fibronectin, which is a commonly used adhesive protein. After washing off unbound fibronectin, the hydrogels were seeded with NIH 3T3 fibroblasts cells. Subsequently, the adhered cells were stained to visualize the cell cytoskeleton and nucleus and imaged by epifluorescence microscopy. Significant cell attachment was observed on the fibronectin-coated hydrogels, whereas cell attachment was minimal on hydrogels incubated with control fibronectin lacking the tetrazine group, which was unable to conjugate to the hydrogel.

Feasibility has been demonstrated for hydrogel synthesis, functionalization with a model protein as well as a commonly used protein in cell culture applications, and cell seeding for conventional culture experiments.

While illustrative embodiments have been illustrated and described, it will be appreciated that various changes can be made therein without departing from the spirit and scope of the disclosure.

1. A method of making hydrogel microspheres, comprising:
(a) providing a reaction mixture comprising:
(i) a biocompatible polymer, wherein the biocompatible polymer comprises at least two of a first reactive group on each polymer, and
(ii) a crosslinker comprising at least two of a second reactive group, wherein the second reactive group is reactive with the first reactive group via a first click reaction;
(b) reacting the reaction mixture to react the first and second reactive groups to provide a crosslinked hydrogel microsphere;
(c) providing a therapeutic agent comprising a third reactive group, wherein the third reactive group is reactive with the first reactive group via a second click reaction; and
(d) reacting the therapeutic agent with the first reactive group in the crosslinked hydrogel microsphere to provide a therapeutic agent-conjugated hydrogel microsphere.

2. The method of claim 1, wherein the reaction mixture is an aqueous two-phase emulsion.

3. The method of claim 2, wherein the aqueous two-phase emulsion comprises a dextran bulk phase and a poly(ethylene glycol) dispersed phase.

4. (canceled)

5. The method of claim 1, wherein the first click reaction is a photoinitiated click reaction.

6. The method of claim 1, wherein the second click reaction is a non-photoinitiated click reaction.

7. The method of claim 1, wherein in (b), further comprising electrospraying the reaction mixture while reacting the reaction mixture.

8. The method of claim 1, wherein in (b), reacting the reaction mixture comprises irradiating the reaction mixture.

9. The method of claim 1, wherein the first reactive group is selected from a norbornene moiety, a substituted norbornene moiety, an aromatic or aliphatic aldehyde moiety, and a C₂₋₆ alkyne moiety.

10. (canceled)

11. The method of claim 9, wherein C₂₋₆ alkyne moiety is a pentyne or propargyl moiety.

12. The method of claim 1, wherein the second reactive group is selected from a thiol moiety, an aminooxy moiety, and an azide moiety.

13-14. (canceled)

15. The method of claim 1, wherein reaction mixture comprises a first reactive group to second reactive group ratio of greater than or wherein in (b), the second reactive groups is reacted with a portion of the first reactive groups to provide a crosslinked hydrogel microsphere comprising unreacted first reactive groups.

16. (canceled)

17. The method of claim 1, wherein the third reactive group is selected from a tetrazine moiety, a hydrazine moiety, and a thiol moiety.

18. (canceled)

19. The method of claim 1, wherein the therapeutic agent is selected from a protein, a peptide, and a small molecule therapeutic agent.

20. The method of claim 1, wherein the biocompatible polymer is selected from poly(ethylene glycol), hyaluronic acid, alginate, and gelatin.

21-22. (canceled)

23. The method of claim 20, wherein the poly(ethylene glycol) is a tetra(poly(ethylene glycol)) comprising terminal norbornene moieties.

24-26. (canceled)

27. A crosslinked hydrogel microparticle comprising poly(ethylene glycol) chains crosslinked with 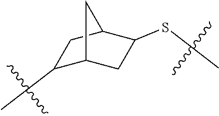 [SMILES: *SC1CC2CC1CC2*]
moieties; and
therapeutic agents-containing moieties of Formula (I)

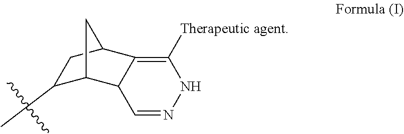

28. A method of making a hydrogel substrate, comprising:
(a) providing a reaction mixture comprising:
(i) a biocompatible polymer, wherein the biocompatible polymer comprises at least two of a first reactive group on each polymer, and
(ii) a crosslinker comprising at least two of a second reactive group, wherein the second reactive group is reactive with the first reactive group via a first click reaction;
(b) reacting the reaction mixture to react the first and second reactive groups to provide a crosslinked hydrogel substrate;
(c) providing an anchor molecule comprising a third reactive group, wherein the third reactive group is reactive with the first reactive group via a second click reaction; and
(d) reacting the anchor molecule with the first reactive group in the crosslinked hydrogel substrate to provide an anchor molecule-functionalized hydrogel substrate,
wherein the anchor molecule is a cell-adhesion molecule.

29-52. (canceled)

53. A crosslinked hydrogel substrate made according to the method of claim 28, comprising poly(ethylene glycol) chains crosslinked with 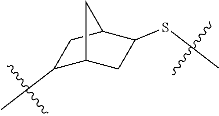 [SMILES: *SC1CC2CC1CC2*]
moieties; and
anchor molecule-containing moieties of Formula (II) 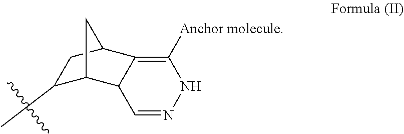
wherein the anchor molecule is a cell-adhesion molecule.

54. A kit, comprising:
(a) a hydrogel substrate made using the method of claim 28, wherein the hydrogel substrate comprises unreacted first reactive groups, and
(b) a cell-adhesion molecule comprising a reactive group selected from a tetrazine moiety, a hydrazine moiety, and a thiol moiety.

55. A kit, comprising:
(a) a hydrogel substrate made using the method of claim 28, wherein the hydrogel substrate comprises unreacted first reactive groups;
(b) an activated ester of 5-(4-(1,2,4,5-tetrazin-3-yl)benzylamino)-5-oxopentanoic acid; and
(c) an amino-group-containing protein or peptide.

56. (canceled)
